(19) United States
(12) Patent Application Publication
(10) Pub. No.: US 2012/0034462 A1
(43) Pub. Date: Feb. 9, 2012

(54) HAIR-LIKE SHAPED HYDROGELS FOR SOFT TISSUE AUGMENTATION

(75) Inventors: Dimitrios Stroumpoulis, Goleta, CA (US); Karina Heredia Guillen, Santa Barbara, CA (US)

(73) Assignee: ALLERGAN, INC., Irvine, CA (US)

(21) Appl. No.: 13/273,888

(22) Filed: Oct. 14, 2011

Related U.S. Application Data
(62) Division of application No. 12/753,361, filed on Apr. 2, 2010.
(60) Provisional application No. 61/166,190, filed on Apr. 2, 2009.

(51) Int. Cl. A61K 9/70; D01F 9/00; C08B 37/08

(52) U.S. Cl. 428/401; 536/55.1

(57) ABSTRACT

Hair-like shaped crosslinked hydrogels and methods for preparing such crosslinked hydrogels and are provided.

### RELATED APPLICATION

This application is a divisional of U.S. patent application Ser. No. 12/753,361 filed Apr. 2, 2010, which claims the benefit of U.S. Provisional Patent Application No. 61/166,190, filed on Apr. 2, 2009, the entire disclosures of which are incorporated herein by reference.

### FIELD OF THE INVENTION

The present invention generally relates to hydrogels useful for soft tissue augmentation, and more specifically relates to methods of making or processing such hydrogels useful for soft tissue augmentation.

### BACKGROUND OF THE INVENTION

Hyaluronic acid (HA), also known as hyaluronan, is a naturally occurring, water soluble polysaccharide, specifically a glycosaminoglycan, which is a major component of the extra-cellular matrix and is widely distributed in animal tissues. HA has excellent biocompatibility and does not cause allergic reactions when implanted into a patient. In addition, HA has the ability to bind large amounts of water, making it an excellent volumizer of soft tissues.

Methods of preparing HA-based soft tissue fillers including both crosslinked and free HA are well known. Crosslinked HA is generally formed by reacting free HA with a crosslinking agent under suitable reaction conditions.

The development of HA-based fillers which exhibit ideal in vivo properties as well as ideal surgical usability has proven difficult. For example, HA-based fillers that exhibit desirable stability properties in vivo, can be so highly viscous that injection through fine gauge needles is difficult or impossible. Conversely, HA-based fillers that are relatively easily injected through fine gauge needles often have inferior stability properties in vivo.

The rate of clearance of an implanted biodegradable material from a location in a body depends on several factors; for example, material shape and size, as well as other mechanisms that can degrade the material into smaller components (e.g. enzymatic or free radical degradation).

Two of the primary clearance mechanisms of implanted biomaterials, for example, implanted HA-based hydrogels used for soft tissue augmentation, are lymphatic drainage and phagocytosis.

Hydrogels intended for soft-tissue augmentation are often formulated to be injectable through a fine gauge needle. This is conventionally accomplished by a process referred to in the industry as “sizing” which generally involves passing a bulk hydrogel material in solid gel form through a sieve multiple times in order to reduce the hydrogel material to micron-sized hydrogel particles which can flow. The hydrogel particles may then be mixed with uncrosslinked HA to improve lubricity in the hydrogel and facilitate its injection through a needle.

### SUMMARY OF THE INVENTION

The present invention provides methods for preparing crosslinked hydrogels for soft tissue augmentation. The present method decreases the extrusion force necessary to extrude crosslinked hydrogels through fine needles and in addition results in hydrogels with higher resistance to lymphatic drainage relative to conventionally prepared hydrogels.

In one embodiment, the method comprises providing a hydrogel material, for example, a crosslinked hydrogel material, for example, a hyaluronic acid based hydrogel material, and forming the material into multiple thin, hydrogel strands, and packaging the product for use as an injectable soft tissue filler while the material is in the form of said multiple thin strands.

In another embodiment, a soft tissue filler is provided wherein the filler comprises a hydrogel a hydrogel product having a strand-like structure. The product may be made by a process comprising the steps of preparing a crosslinked hydrogel material, passing the crosslinked hydrogel material through a mesh, and packaging the hydrogel product for use as a soft tissue filler. The

hydrogel product comprises hydrogel strands generally having diameters of between about 1 μm and about 200 μm, for example, between about 25 μm and about 60 μm and lengths of at least about 0.1 mm up to about 5 mm.

### BRIEF DESCRIPTION OF THE DRAWINGS

FIG. 1 shows extrusion force test results of a HA-based hydrogel product made in accordance with a method of the present invention and a HA-based hydrogel product made in accordance with prior art methods.

FIG. 2 is a chart showing particle affinity of HA hydrogels made in accordance with methods of the present invention and made in accordance with prior art methods.

### DETAILED DESCRIPTION

The present invention provides methods for preparing crosslinked hydrogels for soft tissue augmentation. The present method decreases the extrusion force necessary to extrude crosslinked hydrogels through fine needles and in addition result in hydrogels with higher resistance to lymphatic drainage relative to conventionally prepared hydrogels.

In one embodiment, the method comprises providing a hydrogel material, for example, a crosslinked hydrogel material, for example, a hyaluronic acid based hydrogel material, and forming the material into multiple thin, hydrogel strands, and packaging the product for use as an injectable soft tissue filler while the material is in the form of said multiple thin strands.

In a specific embodiment, the hydrogel material, prior to being formed into multiple thin strands, comprises a solid mass of crosslinked hyaluronic acid based gel. The solid mass may be formed into multiple strands by passing or extruding the solid mass through a sieve or mesh. The sieve or mesh may comprise a mesh having pores or interstices of between about 1 μm and about 200 μm, resulting in strands of material having diameters corresponding to the size of the pores or interstices.

In one aspect of the invention, the hydrogel material is passed or extruded through the sieve or mesh a single time prior to being packaged for use, for example, as a soft tissue filler product. In other words, the strands of hydrogel are not passed through a sieve or mesh a second time, and consequently retain their strand-like, or hair-like configuration during subsequent processing steps, and during injection thereof into a target soft tissue site.

Conventional wisdom in the hydrogel art teaches that a mass of crosslinked hydrogel must be reduced down to very small micron-sized particles in order to facilitate extrusion through a fine gauge needle and to encourage a smooth appearance in the skin at the injection site.

It has been a surprising discovery which goes against this conventional wisdom that the substantially non-particulate, hair-like shape of the present hydrogel product is relatively more resistant to lymphatic drainage and phagocytosis, while at the same time requires a relatively low extrusion force for injection through a fine needle. It is theorized by the present inventors that the hair-like shape of the present hydrogels facilitate extrusion thereof through fine gauge needles, possibly, by enabling the hydrogels to align along the direction of flow during injection.

The present invention is also directed toward a soft tissue filler composition having a hair-like or strand like shape, for example, dermal and subdermal fillers, based on hyaluronic acids (HA) and pharmaceutically acceptable salts of HA, for example, sodium hyaluronate (NaHA). As used herein, hyaluronic acid (HA) can refer to any of its hyaluronate salts, and includes, but is not limited to, sodium hyaluronate (NaHA), potassium hyaluronate, magnesium hyaluronate, calcium hyaluronate, and combinations thereof.

Generally, the concentration of HA in the present compositions described herein is preferably at least 10 mg/mL and up to about 40 mg/mL. For example, the concentration of HA in some of the compositions is in a range between about 20 mg/mL and about 30 mg/mL. Further, for example, in some embodiments, the compositions have a HA concentration of about 22 mg/mL, about 24 mg/mL, about 26 mg/mL, or about 28 mg/mL.

The compositions comprise a crosslinked HA-based gel product for injection into soft tissue, wherein the product comprises a HA-composition having a strand-like or hair-like shape. In other words, rather than being spherical or particulate in nature when initially injected into soft tissue, the present hydrogel material comprises multiple thin strands of crosslinked hydrogel material.

In some embodiments, the strands have diameters of between about 1 μm and about 200 μm and lengths of at least twice, for example, up to 100 times or greater, than a corresponding diameter. In some embodiments, the strands have a diameter of between about 25 μm and about 60 μm, and lengths of between about 100 μm up to several mm, for example up to about 5 mm. The strands may have a generally square, round, angular or other cross sectional shape, which in some embodiments, depends on the technique for forming the strands from the initial gel. For example, the strands may have cross-sectional shaped substantially conforming to the shape of the pores in a sieve used to form the strands from the initial gel.

Strand length may be somewhat dependent on the cohesivity of the HA composition used to form the strands. Although not intending to be bound by any particular theory of operation, it is hypothesized by the present inventors that gels having relatively high cohesivity will produce longer strands while gels having relatively low cohesivity produce shorter strands. It is believed that gels with lower cohesivity are relatively more brittle and thus break to form smaller strands.

Further described herein is a method for preparing HA-based compositions having a strand-like or hair-like shape by preparing a precursor composition, for example, a cohesive, crosslinked HA-based gel and passing the gel through a sieve, mesh or other device to obtain the desired structure. In some embodiments, the gel is passed through a sieve or mesh only one time prior to it being used as an injectable product.

In certain embodiments, the precursor composition is a cohesive, hydrated HA-based gel. Such a gel will generally include no greater than between about 1% to about 10% soluble-liquid form or free HA by volume. In certain embodiments, less than about 1% to about 10% of the precursor composition comprises free (i.e. uncrosslinked or lightly crosslinked) HA.

In yet other embodiments, the precursor composition is a relatively non-cohesive, hydrated HA-based gel. Such a “non-cohesive” gel generally includes greater than 10%, for example, greater than about 15%, for example, greater than 20% or more of free HA.

In some embodiments, the precursor composition may comprise a first component made up of relatively highly crosslinked HA in a substantially solid phase, and a second component comprising free or relatively less crosslinked HA in a substantially fluidic phase in which the relatively highly crosslinked HA is dispersed.

In some embodiments, the present soft tissue filler compositions made from the above mentioned precursor compositions, have a somewhat strand-like nature as described elsewhere herein. The compositions comprise elongated strands of relatively highly crosslinked HA, dispersed in a medium of free HA.

The strands generally have a substantially uniform diameter and a length that is at least two times, for example, at least three times, for example, at least ten times, for example, at least 20 times, for example, at least 50 times, for example, at least 100 times or greater, than a corresponding diameter of the strands. In some embodiments, the average diameter of such strands of crosslinked HA is about 1 μm, for example, about 100 μm, for example about 200 μm or about 250 μm.

The precursor composition may be manufactured by pressing a mass of crosslinked HA-based gel through a sieve or a mesh to create crosslinked HA strands of generally uniform size and shape. These strands may then be mixed with a carrier material, for example, an amount of free HA, to produce a gel product that can be used as an effective soft tissue filler, for example, a facial filler. The gel product is relatively easily extruded through a fine gauge needle in that less force may be required for the extrusion, for example, relative to a substantially identical gel that does not have such a strand-like structure. In some embodiments, the gel product resists degradation, after being placed in the patient, more readily relative to a substantially identical gel that does not have such a strand like structure.

Manufacturing of the present HA compostions may comprise, in one embodiment, the initial step of providing raw HA material in the form of dry HA fibers or powder. The raw HA material may be HA, its salts and/or mixtures thereof. The HA material may comprise fibers or powder of NaHA, and in some embodiments, bacterial-sourced NaHA. Alternatively, the raw HA material may be animal derived. The HA material may be a combination of raw materials including HA and at least one other polysaccharide, for example, glycosaminoglycan (GAG).

In some embodiments, the HA material in the compositions nearly entirely comprises or consists of high molecular weight HA. That is, nearly 100% of the HA material in the present compositions may be high molecular weight HA as defined below. In other embodiments, the HA material in the compositions comprises a combination of relatively high molecular weight HA and relatively low molecular weight HA, as defined below.

High molecular weight HA as used herein describes a HA material having a molecular weight of at least about 1.0 million Daltons (mw≧10⁶ Da or 1 MDa) to about 4.0 MDa. For example, the high molecular weight HA in the present compositions may have a molecular weight of about 2.0 MDa. In another example, the high molecular weight HA may have a molecular weight of about 2.8 MDa.

Low molecular weight HA as used herein describes a HA material having a molecular weight of less than about 1.0 MDa. Low molecular weight HA can have a molecular weight of between about 200,000 Da (0.2 MDa) to less than about 1.0 MDa, for example, between about 300,000 Da (0.3 M Da) to about 750,000 Da. (0.75 MDa).

The HA material of the compositions may comprise between about 5% to about 95% high molecular weight HA with the balance of the HA material including low molecular weight HA. In one embodiment of the invention, the ratio of high molecular weight to low molecular weight HA is at least about, and preferably greater than 2 (w/w≧2) with the high molecular weight HA having a molecular weight of above 1.0 MDa.

It will be appreciated by those of ordinary skill in the art that the selection of high and low molecular weight HA material and their relative percentages or ratios is dependent upon the desired characteristics, for example, extrusion force, elastic modulus, viscous modulus and phase angle expressed as the ratio of viscous modulus to elastic modulus, cohesivity, etc. of the final HA-based product.

The HA-based gels can be prepared according to the present invention by first cleaning and purifying the dry or raw HA material having a desired high/low molecular weight ratio. These steps generally involve hydrating the dry HA fibers or powder in the desired high/low molecular weight ratio, for example, using pure water, and filtering the material to remove large foreign matters and/or other impurities. The filtered, hydrated material is then dried and purified. The high and low molecular weight HA may be cleaned and purified separately, or may be mixed together, for example, in the desired ratio, just prior to crosslinking.

In accordance with the present invention, pure, dry NaHA fibers are hydrated in an aqueous solution, for example, a neutral, slightly acidic or alkaline solution, to produce a free NaHA gel. In one embodiment, a suitable alkaline solution may be used to hydrate the NaHA, for example, but not limited to aqueous solutions containing sodium hydroxide (NaOH), potassium hydroxide (KOH), sodium bicarbonate (NaHCO₃), lithium hydroxide (LiOH), and the like. In another embodiment, the suitable alkaline solution is aqueous solutions containing NaOH. The resulting alkaline gel will have a pH above 7.5. The pH of the resulting alkaline gel can have a pH greater than 9, or a pH greater than 10, or a pH greater than 12, or a pH greater than 13.

The manufacturing process further involves the step of crosslinking the hydrated NaHA gel with a suitable crosslinking agent. The crosslinking agent may be any agent known to be suitable for crosslinking polysaccharides and their derivatives via their hydroxyl groups. Suitable crosslinking agents include, but are not limited to, 1,4-butanediol diglycidyl ether (or 1,4-bis(2,3-epoxypropoxy)butane or 1,4-bisglycidyloxybutane, all of which are commonly known as BDDE), 1,2-bis(2,3-epoxypropoxy)ethylene and 1-(2,3-epoxypropyl)-2,3-epoxycyclohexane. The use of more than one crosslinking agent or a different crosslinking agent is not excluded from the scope of the present invention. In one embodiment, the HA gels described herein are crosslinked using BDDE.

The step of crosslinking may be carried out using any means known to those of ordinary skill in the art. Those skilled in the art appreciate how to optimize conditions of crosslinking according to the nature of the HA, and how to carry out crosslinking to an optimized degree. Degree of crosslinking for purposes of the present invention is defined as the percent weight ratio of the crosslinking agent to HA-monomeric units within the crosslinked portion of the HA based composition. It is measured by the weight ratio of HA monomers to crosslinker (HA monomers:crosslinker).

In some embodiments, the HA is crosslinked during the step of hydration of the raw HA fibers. In other embodiments the HA is crosslinked after the step of hydration of the raw HA fibers.

The degree of crosslinking in the HA component of the present compositions is at least about 2% and is up to about 20%. In other embodiments, the degree of crosslinking is greater than 5%, for example, is about 6% to about 8%. In some embodiments, the degree of crosslinking is between about 4% to about 12%. In some embodiments, the degree of crosslinking is less than about 6%, for example, is less than about 5%.

In some embodiments, the HA gel is capable of absorbing at least about one time its weight in water. When neutralized and swollen, the crosslinked HA component and water absorbed by the crosslinked HA component is in a weight ratio of about 1:1.

Once the HA gel is made by mixing the desired high and low molecular weight ratios of dry HA fibers, hydrating the dry fibers and crosslinking the HA component to the desired degree, the next step of the present invention involves shaping or forming the strand-like hydrogels. Shaping or forming of the strand-like hydrogels may be accomplished by passing the crosslinked HA gel mass through a mesh, screen sieve, or other suitable mechanism to cut through the mass of gel and thereby form the strand-like shaped hydrogels therefrom. In accordance with a particular embodiment, the strand-like hydrogels are not subjected to any further cutting, shaping or sizing steps. In one embodiment, the precursor HA gel is passed through a mesh, sieve or screen only one time prior to the final product being packaged in a syringe for use as a soft tissue filler. It is contemplated that this shaping or forming step may, in some instances, be repeated in accordance with other embodiments of the invention, so long as the resulting hydrogels retain their strand-like shape.

### Example 1

### Preparation of a HA Soft Tissue Filler Product According to the Present Invention

1 gram of sodium hyaluronate fibers (NaHA, Mw=0.5-3 MDa) is mixed with 5-10 g of 1% sodium hydroxide solution and the mixture is allowed to hydrate for 1-5 hrs forming a hydrated NaHA gel. 50-200 mg of 1,4-butanediol diglycidyl ether (BDDE) are added to the NaHA gel and the mixture is mechanically homogenized.

The mixture is then placed in a 40-70° C. oven for 1-4 hrs. The resulting cross-linked hydrogel is neutralized with an equimolar amount of hydrochloric acid (HCl) and swelled in phosphate buffered saline, (PBS, pH 7). The hydrogel is sized by passing it through a 25 μm or 43 μm mesh screen one (1) time. After being passed through the mesh screen a single time, the resulting thin, hair-like strands of hydrogel are dialyzed, packaged and sterilized.

### Example 2

### Preparation of a HA Filling Gel by the Process of the Prior Art

1 gram of sodium hyaluronate fibers (NaHA, Mw=0.5-3 MDa) is mixed with 5-10 g of 1% sodium hydroxide solution and the mixture is allowed to hydrate for 1-5 hrs. 50-200 mg of 1,4-butanediol diglycidyl ether (BDDE) are added to the NaHA gel and the mixture is mechanically homogenized.

The mixture is then placed in a 40-70° C. oven for 1-4 hrs. The resulting cross-linked hydrogel is neutralized with an equimolar amount of hydrochloric acid (HCl) and swelled in PBS (pH 7). The hydrogel is sized by passing it through a 105 μm mesh screen seven (7) times. After being passed through the mesh screen seven times, the resulting micron-sized hydrogel particles are dialyzed, packaged and sterilized.

### Comparison 1

### Continuous Extrusion Force Test

To evaluate the rheological properties of the HA filling gels prepared in Examples 1 and 2, continuous extrusion force tests were performed. This test measures the force needed to pass the gel through a needle. Specifically, the lower the extrusion force, the easier it is to extrude a gel. Extrusion forces less than 40 N through a 30 G needle are desirable for injection into soft tissue.

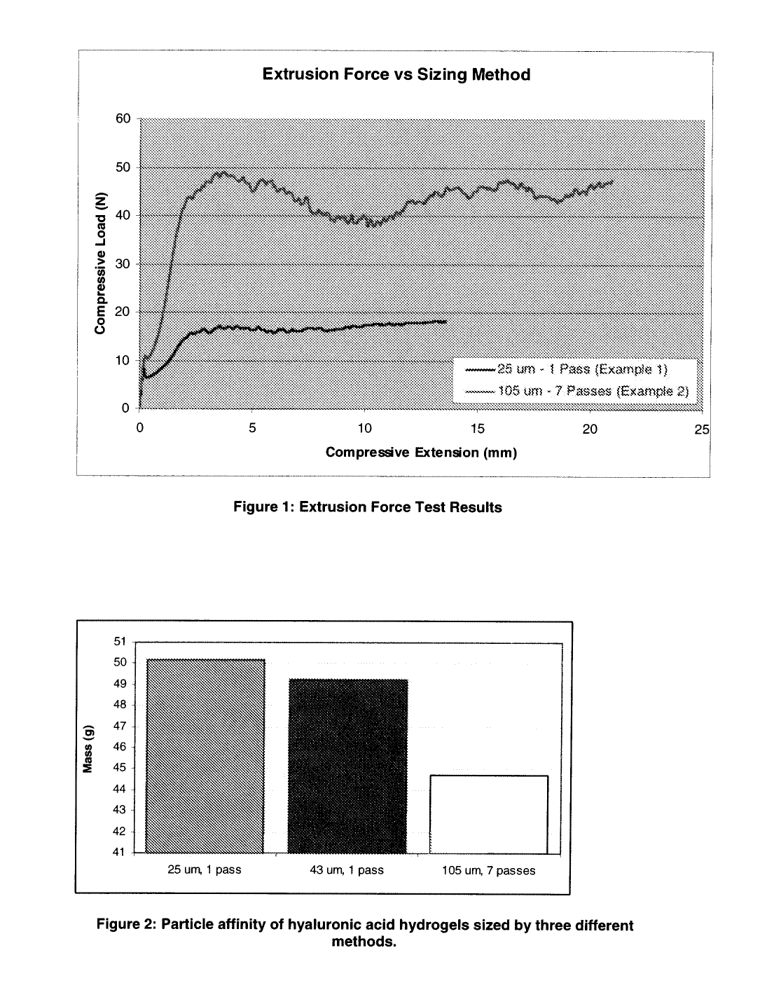

The extrusion force tests were performed on an Instron instrument using a 1 mL syringe with a 27 G needle. 0.5 mL of each sample was extruded at a constant rate of 50 mm/min. The peak force recorded quantifies the ease of extrusion. The compressive force as a function of the compressive extension for the two samples is plotted in FIG. 1. The results show that the extrusion force peak recorded for the gel prepared by the process of the invention is significantly lower than that recorded for the process of the prior art. Further, the extrusion force profile for the former case is smoother as demonstrated by a relatively flat plateau.

### Comparison 2

### Particle Affinity

To assess the cohesivity of the gels, particle affinity measurements were performed. This assay indirectly measures the affinity the gel has for itself by measuring the mass of 5 gel droplets formed while extruding through a 30 gauge needle at a constant rate. A gel with a higher particle affinity (i.e. more cohesive/sticky) will have larger and heavier droplets. Three gels were synthesized as described above, and sized by three different methods. The first method was via 1 pass through a 25 μm mesh and the second was passed 1 time through a 43 μm mesh, forming hair-like gel. The third sizing method was performed by passing the gel 7 times through a 105 μm mesh. This results in a particulate gel. Shown in FIG. 2 are the particle affinity results. The gels passed 1 time through the 25 and 43 μm mesh, have higher particle affinities than the particulate gel formed from multiple passes through the 105 μm mesh.

### Example 3

NaHA fibers or powder are hydrated in an alkaline solution, for example, an aqueous solution containing NaOH. The mixture is mixed at ambient temperature, about 23° C., to form a substantially homogenous, alkaline HA gel.

A crosslinking agent, BDDE, is diluted in an aqueous solution and added to the alkaline HA gel. The mixture is homogenized for several minutes.

Alternatively, BDDE can be added directly to the HA fibers (dry state) at the beginning of the process, prior to the hydration. The crosslinking reaction will then start relatively slowly at ambient temperature, ensuring even better homogeneity and efficacy of the crosslinking. Methods of crosslinking polymers in the dry state using a polyfunctional crosslinking agent such as BDDE are described in, for example, Piron et al., U.S. Pat. No. 6,921,819 which is incorporated herein by reference in its entirety as if it were part of the present specification.

The resulting crosslinked HA gel mixture is then heated at about 50° C. for about 2.5 hours. The material is now a highly crosslinked HA/BDDE gel (aspect=solid gel). This crosslinked gel is then neutralized with a suitable acidic solution. The neutralized HA gel is then swollen in a phosphate buffer at a cold temperature, for example a temperature of about 5° C., to obtain a highly cohesive HA gel. In this specific example, the phosphate buffered saline solution contains water-for-injection (WFI), disodium hydrogen phosphate, and sodium dihydrogen phosphate. When neutralized and swollen, the crosslinked HA component and water absorbed by the crosslinked HA component is in a weight ratio of about 1:1. The hydrogel is then passed through a mesh screen one (1) time (screen pore diameter 25 μm-60 μm) creating a HA gel comprising hair-like strands having diameters about equivalent to the screen pore diameter and lengths generally between about 0.5 mm and about 3 mm.

The hair-like HA gel is then mechanically stirred and filled into dialysis membranes and dialyzed against a phosphate buffer. The gel is then filled into dialysis membranes and dialyzed against a phosphate buffer for up to several days with regular changes of the bath, in order to remove the un-reacted crosslinker, to stabilize the pH close to neutrality (pH=7.2) and to ensure proper osmolarity of the HA gel. The gel is then packaged into syringes for dermal injection and sterilized in accordance with conventional means.

### Example 4

The hair-like shaped HA gels is made in accordance with the method described in EXAMPLE 3, except that prior to packaging into syringes, lidocaine chlorhydrate (lidocaine HCl) is added. First, lidocaine HCl in powder form is first solubilized in WFI and filtered through a 0.2 μm filter. Dilute NaOH solution is added to the HA gel in order to reach a slightly basic pH (for example, a pH of between about 7.5 and about 8). The lidocaine HCl solution is then added to the slightly basic gel to reach a final desired concentration, for example, a concentration of about 0.3% (w/w). The resulting pH of the HA/lidocaine mixture is then about 7 and the HA concentration is about 24 mg/mL. Mechanical mixing may be performed in order to obtain a proper homogeneity.

If desired, a suitable amount of free HA gel may be added to the HA/lidocaine gel mixture with the advantage of increasing the kinetics of lidocaine delivery. For example, free HA fibers are swollen in a phosphate buffer solution, in order to obtain a homogeneous viscoelastic gel. This free HA gel is then added to the crosslinked HA/lidocaine gel (for example, at about 5%, w/w). The resulting gel is then filled into sterile syringes and autoclaved at sufficient temperatures and pressures for sterilization for at least about 1 minute.

After autoclaving, the final HA/lidocaine product is packaged and distributed to physicians. The product manufactured in accordance with this method exhibits one or more characteristics of stability as defined elsewhere herein. For example, the autoclaved HA/lidocaine product has a viscosity, cohesivity, and extrusion force that are acceptable. No degradation of the HA/lidocaine gel product is found during testing of the product after the product has spent several months in storage.

While this invention has been described with respect to various specific examples and embodiments, it is to be understood that the invention is not limited thereto and that it can be variously practiced within the scope of the invention.

What is claimed is:

1. A soft tissue filler composition comprising:
a crosslinked HA-based gel comprising a multiple strands having a thickness of about 1-200 μm and varying lengths.

2. A soft tissue filler comprising a hydrogel product having a strand-like structure.

3. The filler of claim 2 wherein the hydrogel product is made by a process comprising the steps of preparing a crosslinked hydrogel material and passing the crosslinked hydrogel material through a mesh to obtain the hydrogel product.

4. The filler of claim 2 wherein the hydrogel product is made by a process comprising the steps of preparing a crosslinked hydrogel material and passing the crosslinked hydrogel material through a mesh only one time to obtain the hydrogel product.

5. The filler of claim 2 wherein the hydrogel product is made by a process further comprising the step of packaging the hydrogel product for use as a soft tissue filler without passing the crosslinked hydrogel through a mesh an additional time prior to said packaging.

6. The filler of claim 2 wherein the hydrogel product comprises hydrogel strands having diameters of between about 1 μm and about 200 μm.

7. The filler of claim 2 wherein the hydrogel product comprises hydrogel strands having diameters of between about 1 μm and about 60 μm.

8. The filler of claim 2 wherein the hydrogel product comprises hydrogel strands having diameters of between about 25 μm and about 60 μm.

9. The filler of claim 2 wherein the hydrogel product comprises hydrogel strands having lengths of at least about 0.1 mm.

10. The filler of claim 2 wherein the hydrogel product comprises hydrogel strands having lengths of between about 0.1 mm and about 5 mm.

11. The filler of claim 2 wherein the hydrogel product comprises hydrogel strands having lengths of between about 0.5 mm and about 3 mm.

12. The filler of claim 2 wherein the hydrogel product comprises hydrogel strands having diameters of between about 25 μm and about 60 μm and lengths of between about 0.1 mm and about 5 mm.

13. The filler of claim 12 wherein the hydrogel product is made by a process comprising the steps of preparing a crosslinked hydrogel material and passing the crosslinked hydrogel material through a mesh to obtain the hydrogel product.

14. The filler of claim 12 wherein the hydrogel product is made by a process comprising the steps of preparing a crosslinked hydrogel material and passing the crosslinked hydrogel material through a mesh only one time to obtain the hydrogel product.

15. The filler of claim 12 wherein the hydrogel product is made by a process further comprising the step of packaging the hydrogel product for use as a soft tissue filler without passing the crosslinked hydrogel through a mesh an additional time prior to said packaging.
